(19) United States
(12) Patent Application Publication
(10) Pub. No.: US 2021/0284956 A1
(43) Pub. Date: Sep. 16, 2021

(54) EUGLENA CULTURE MEDIUM AND APPLICATION THEREOF

(71) Applicant: Youge Tiancheng Biotechnology (Yiwu) Co., Ltd., Yiwu City (CN)

(72) Inventors: Jiangxin Wang, Yiwu City (CN); Mingcan Wu, Yiwu City (CN); Jing Li, Yiwu City (CN)

(73) Assignee: Youge Tiancheng Biotechnology (Yiwu) Co., Ltd., Yiwu City (CN)

(21) Appl. No.: 16/991,576

(22) Filed: Aug. 12, 2020

(30) Foreign Application Priority Data
Mar. 12, 2020 (CN) 202010169960.6

(51) Int. Cl. C12N 5/00

(52) U.S. Cl. CPC C12N 5/0025; C12N 2500/24; C12N 2500/16

(57) ABSTRACT

The present invention belongs to the field of biotechnology, and particularly relates to a Euglena culture medium and an application thereof. The Euglena culture medium includes the following components: NH₄Cl, KH₂PO₄, MgSO₄.7H₂O, CaCl₂.2H₂O, Na₂EDTA.2H₂O, Fe₂(SO₄)₃, CuSO₄.5H₂O, ZnSO₄.7H₂O, Co.(NH₃).H₂O, MnCl₂.4H₂O, vitamin B₁ and vitamin B₁₂. The pH is adjusted based on the Euglena culture medium of the present invention to achieve the purpose of preconcentrating Euglena cells. In the present invention, the Euglena cells are less ruptured, which improves the concentration efficiency of Euglena cells and reduces the treatment cost.

### CROSS-REFERENCE TO RELATED APPLICATIONS

This application claims priority under 35 U.S.C. 119 to the Chinese Application No. 202010169960.6, filed Mar. 12, 2020, the entirety of which is incorporated by reference herein.

### TECHNICAL FIELD

The present invention belongs to the field of biotechnology, and particularly relates to a Euglena culture medium and an application thereof.

### BACKGROUND

As a single-celled, cell-wall-free and flagellate microalgae, Euglena has a wide range of uses in carbon emission reduction, food additives, natural pigments, unsaturated fatty acids, and biofuels, etc. However, since the diameter of Euglena cells is about 5-30 μm and the biomass is only in the range of 0.5-2 g/L under photoautotrophic conditions, it is difficult to harvest Euglena cells. If Euglena cells are directly concentrated using a centrifuge, the production cost will be increased. Therefore, a method for pre-concentrating Euglena cells is needed so as to reduce the production cost.

At present, the microalgae pre-concentration methods include electrolytic method, air flotation method, gravity precipitation method, ultrafiltration (UF) membrane method and flocculation method. Among them, the more economical is the flocculation method.

The conventional algal cell culture medium must adjust the pH to above 10.5 to flocculate and precipitate the algal cells to achieve the pre-concentration purpose. However, an excessively high pH will cause the algal cells to rupture or destroy the active substances of the algal cells due to excessive osmotic pressure, thereby reducing the pre-concentration efficiency. An excessively high pH will also have an adverse impact on the wastewater discharge standards, resulting in the need to increase the acid feed to restore the original pH, thereby increasing the treatment cost.

### SUMMARY

An objective of the present invention is to provide a Euglena culture medium for pre-concentrating Euglena cells with high efficiency. The Euglena culture medium provided by the present invention improves the concentration efficiency of Euglena cells.

To achieve the above purpose, the present invention provides the following technical solutions.

A Euglena culture medium includes the following components: NH₄Cl, KH₂PO₄, MgSO₄.7H₂O, CaCl₂.2H₂O, Na₂EDTA.2H₂O, Fe₂(SO₄)₃, CuSO₄.5H₂O, ZnSO₄.7H₂O, Co.(NH₃).H₂O, MnCl₂.4H₂O, vitamin B₁ and vitamin B₁₂, and the pH of the Euglena culture medium is 8-10.

Preferably, the Euglena culture medium includes the following components:1.5-2.7 g/L NH₄Cl, 0.6-2.4 g/L KH₂PO₄, 1.2-2.4 g/L MgSO₄.7H₂O, 0.02-0.10 g/L CaCl₂.2H₂O, 0.55-0.78 μg/L Na₂EDTA.2H₂O, 2-4 μg/L Fe₂(SO₄)₃, 0.05-0.08 μg/L CuSO₄.5H₂O, 0.4-0.7 μg/L ZnSO₄.7H₂O, 1.2-1.5 μg/L Co.(NH₃).H₂O, 1.8-2.2 μg/L MnCl₂.4H₂O, 0.01-0.09 μg/L vitamin B₁ and 0.0005-0.0015 μg/L vitamin B₁₂.

The present invention further provides an application of the above Euglena culture medium in Euglena pre-concentration.

Preferably, the Euglena includes Euglena gracilis.

Preferably, the application includes the following steps:

(1) inoculating Euglena into the Euglena culture medium to obtain a secondary Euglena solution;

(2) transferring the secondary Euglena solution to a photobioreactor, and continuing culture to obtain a Euglena solution; and

(3) adjusting pH of the Euglena solution to 8-10, and leaving the Euglena solution to stand to obtain pre-concentrated Euglena.

Preferably, in step (3), a pH adjusting agent includes 2-4 M of sodium hydroxide and 0.5-1.5 M of hydrochloric acid, and the standing time is preferably 60-120 min.

Preferably, in step (2), the culture is performed under a condition with a light intensity of 100-200 μmol photons m⁻²·s⁻¹ and an aeration rate of 3-7 L/min.

Preferably, a gas used to maintain the aeration rate includes carbon dioxide; the volume content of the carbon dioxide in the gas is 1-3%; the carbon dioxide is carbon dioxide filtered through a 0.2 μm membrane.

Preferably, in step (2), the culture time is 6-8 d.

Preferably, in step (1), the culture is performed under a sterile condition at 20-30° C. and a light intensity of 30-70 μmol photons m⁻²·s⁻¹.

The present invention provides a Euglena culture medium, including the following components: NH₄Cl, KH₂PO₄, MgSO₄.7H₂O, CaCl₂).2H₂O, Na₂EDTA.2H₂O, Fe₂(SO₄)₃, CuSO₄.5H₂O, ZnSO₄.7H₂O, Co.(NH₃).H₂O, MnCl₂.4H₂O, vitamin B₁ and vitamin B₁₂, where the pH of the Euglena culture medium is 8-10. The culture medium of the present invention uses various components to provide nutrients for Euglena. On this basis, magnesium and calcium ions provided by MgSO₄.7H₂O and CaCl₂) further produce magnesium phosphate and calcium phosphate precipitates, which in turn lead to flocculation. By using the Euglena culture medium of the present invention to concentrate a Euglena solution, Euglena is less ruptured, which improves the concentration efficiency of Euglena cells and reduces the treatment cost.

### BRIEF DESCRIPTION OF DRAWINGS

FIG. 1 is a morphological diagram of Euglena cells after treatment with a Euglena culture medium with different pH.

FIG. 2 shows precipitation of Euglena pre-concentrated by different treatments after standing for 60 min.

FIGS. 3A and 3B respectively show Mg²⁺ and Ca²⁺ concentrations in a Euglena solution pre-concentrated by Treatment 1 and Treatment 2.

FIG. 4 shows flocculation of a Euglena culture medium with pH 8.5 after standing for 120 min.

### DETAILED DESCRIPTION

The present invention provides a Euglena culture medium, including the following components: NH₄Cl, KH₂PO₄, MgSO₄.7H₂O, CaCl₂).2H₂O, Na₂EDTA.2H₂O, Fe₂(SO₄)₃, CuSO₄.5H₂O, ZnSO₄.7H₂O, Co.(NH₃).H₂O, MnCl₂.4H₂O, vitamin B₁ and vitamin B₁₂, where the pH of the Euglena culture medium is 8-10.

The Euglena culture medium provided by the present invention preferably includes 1.5-2.7 g/L NH₄Cl, more preferably 1.6-2.3 g/L, and most preferably 1.8 g/L. The present invention has no special limit on the source of the NH₄Cl, and conventional NH₄Cl in the art can be used. NH₄Cl provides nitrogen for the growth and reproduction of Euglena.

The Euglena culture medium provided by the present invention preferably includes 0.6-2.4 g/L KH₂PO₄, more preferably 0.6-2.0 g/L, and most preferably 0.6 g/L. The present invention has no special limit on the source of the KH₂PO₄, and conventional KH₂PO₄ in the art can be used. KH₂PO₄ provides potassium and phosphorus for the growth and reproduction of Euglena, and buffers the pH of the culture medium.

The Euglena culture medium provided by the present invention preferably includes 1.2-2.4 g/L MgSO₄.7H₂O, more preferably 1.2-2.0 g/L, and most preferably 1.2 g/L. The present invention has no special limit on the source of the MgSO₄.7H₂O, and conventional MgSO₄.7H₂O in the art can be used. MgSO₄.7H₂O provides magnesium for the growth and reproduction of Euglena, and a magnesium ion accelerates the flocculation of Euglena.

The Euglena culture medium provided by the present invention preferably includes 0.02-0.10 g/L CaCl₂), more preferably 0.02-0.08 g/L, and most preferably 0.02 g/L. The present invention has no special limit on the source of the CaCl₂).2H₂O, and conventional CaCl₂).2H₂O in the art can be used. CaCl₂).2H₂O provides calcium for the growth and reproduction of Euglena, and a calcium ion causes flocculation of Euglena at a low pH.

The Euglena culture medium provided by the present invention preferably includes 0.55-0.78 g/L Na₂EDTA.2H₂O, more preferably 0.55-0.70 g/L, and most preferably 0.55 g/L. The present invention has no special limit on the source of the Na₂EDTA.2H₂O, and conventional Na₂EDTA.2H₂O in the art can be used. Na₂EDTA.2H₂O provides sodium for the growth and reproduction of Euglena.

The Euglena culture medium provided by the present invention preferably includes 2-4 g/L Fe₂(SO₄)₃, more preferably 2-3 g/L, and most preferably 2 g/L. The present invention has no special limit on the source of the Fe₂(SO₄)₃, and conventional Fe₂(SO₄)₃ in the art can be used. Fe₂(SO₄)₃ provides iron for the growth and reproduction of Euglena.

The Euglena culture medium provided by the present invention preferably includes 0.05-0.08 μg/L CuSO₄.5H₂O, more preferably 0.05-0.07 μg/L, and most preferably 0.05 μg/L. The present invention has no special limit on the source of the CuSO₄.5H₂O, and conventional CuSO₄.5H₂O in the art can be used. CuSO₄.5H₂O provides copper for the growth and reproduction of Euglena.

The Euglena culture medium provided by the present invention preferably includes 0.04-0.07 μg/L ZnSO₄.7H₂O, more preferably 0.04-0.06 μg/L, and most preferably 0.04 μg/L. The present invention has no special limit on the source of the ZnSO₄.7H₂O, and conventional ZnSO₄.7H₂O in the art can be used. ZnSO₄.7H₂O provides zinc for the growth and reproduction of Euglena.

The Euglena culture medium provided by the present invention preferably includes 1.2-1.5 μg/L Co.(NH₃).H₂O, more preferably 1.3-1.4 μg/L, and most preferably 1.3 μg/L. The present invention has no special limit on the source of the Co.(NH₃).H₂O, and conventional Co.(NH₃).H₂O in the art can be used. Co.(NH₃).H₂O provides cobalt and nitrogen for the growth and reproduction of Euglena.

The Euglena culture medium provided by the present invention preferably includes 1.8-2.2 μg/L MnCl₂.4H₂O, more preferably 1.8-2.0 μg/L, and most preferably 1.8 μg/L. The present invention has no special limit on the source of the MnCl₂.4H₂O, and conventional MnCl₂.4H₂O in the art can be used. MnCl₂.4H₂O provides manganese and chlorine for the growth and reproduction of Euglena.

The Euglena culture medium provided by the present invention preferably includes 0.01-0.09 μg/L vitamin B₁, more preferably 0.01-0.06 μg/L, and most preferably 0.01 μg/L. The present invention has no special limit on the source of the vitamin B₁, and conventional vitamin B₁ in the art can be used. The vitamin B₁ provides a nutrient for the growth and reproduction of Euglena.

The Euglena culture medium provided by the present invention preferably includes 0.0005-0.0015 μg/L vitamin B₁₂, more preferably 0.0005-0.0010 μg/L, and most preferably 0.0005 μg/L. The present invention has no special limit on the source of the vitamin B₁₂, and conventional vitamin B₁₂ in the art can be used. The vitamin B₁₂ provides a nutrient for the growth and reproduction of Euglena.

In the present invention, the pH of the Euglena culture medium is 8-10, preferably 8.2-9.5, and more preferably 8.5-9. The culture medium of the present invention uses various components to provide nutrients for Euglena. On this basis, magnesium and calcium ions provided by MgSO₄.7H₂O and CaCl₂).2H₂O further produce magnesium phosphate and calcium phosphate precipitates, which in turn lead to flocculation of a Euglena cell at the pH of 8-10, improving the pre-concentration efficiency of Euglena.

The present invention further provides an application of the Euglena culture medium described in the above technical solution in Euglena pre-concentration.

The Euglena preferably includes Euglena gracilis.

In the present invention, the application of the Euglena culture medium in Euglena pre-concentration preferably includes the following steps:

(1) inoculate Euglena into the Euglena culture medium to obtain a secondary Euglena solution;

(2) transfer the secondary Euglena solution to a photobioreactor, and continue culture to obtain a Euglena solution; and

(3) adjust the pH of the Euglena solution to 8-10, and leave the Euglena solution to stand to obtain pre-concentrated Euglena.

The present invention inoculates Euglena into the Euglena culture medium to obtain a secondary Euglena solution. In the present invention, the culture is preferably performed under a 25° C. sterile condition with a light intensity of 50 μmol photons m⁻²·s⁻¹. In the present invention, the culture is preferably performed in an Erlenmeyer flask; the culture preferably includes: inoculate Euglena into 20 mL of Euglena culture medium, and culture for 7 d to obtain a primary Euglena solution; inoculate the primary Euglena solution into 500 mL of Euglena culture medium, and culture for 7 d to obtain a secondary Euglena solution. The Euglena is inoculated from a solid plate into the liquid medium. The number of Euglena cells at an initial phase is limited, so the volume of the medium may not be too large. After Euglena is inoculated into 20 mL of Euglena culture medium, the number of Euglena cells is increased for further propagation. This step is designed to further increase the concentration of the Euglena cells and the volume of the Euglena solution.

After the secondary Euglena solution is obtained, the present invention transfers the secondary Euglena solution to a photobioreactor to culture to obtain a Euglena solution. In the present invention, the culture is preferably performed under a condition with a light intensity of preferably 100-200 μmol photons m⁻²·s⁻¹ and an aeration rate of preferably 3-7 L/min, and more preferably 5 L/min. The specific culture condition is intended to stabilize the propagation. The culture preferably further includes: introduce carbon dioxide; the volume content of the carbon dioxide is preferably 2%, and the carbon dioxide is preferably carbon dioxide filtered through a 0.2 μm membrane. The carbon dioxide with this concentration is introduced to promote the division of Euglena cells and increase the concentration of Euglena cells. The culture time is preferably 6-8 d, more preferably 7 d.

The culture of the Euglena cells reach a plateau phase in 6-8 d, and the concentration of the Euglena cells no longer increases. It is suitable to harvest Euglena cells in the plateau phase. This step aims to expand the volume of the Euglena solution. In the present invention, the culture is specifically performed in a photobioreactor. The photobioreactor has a stable light system and aeration system, which allows Euglena cells to propagate under a controlled culture condition.

After the Euglena solution is obtained, the present invention adjusts the pH of the Euglena solution to 8-10, and leaves the Euglena solution to stand to obtain a flocculated precipitate, which is pre-concentrated Euglena. In the present invention, the standing time is preferably 60-180 min; a pH adjusting agent is preferably 2-4 M of sodium hydroxide and 0.5-1.5 M of hydrochloric acid, and more preferably 3 M of sodium hydroxide and 1 M of hydrochloric acid. The adding of the sodium hydroxide should not be too fast, so as to avoid a local pH of the Euglena solution being excessively high to cause the Euglena cell to easily rupture.

After the pre-concentrated Euglena is obtained, the present invention preferably centrifuges the pre-concentrated Euglena to further reduce the water content and achieve the concentration of the Euglena cells. In the present invention, the centrifugation is further preferably a high-speed centrifugation, the centrifugal force is preferably 3000×g, the centrifugation time is preferably 5 min, and the centrifugation is specifically performed in a centrifuge.

The Euglena culture medium and the application thereof provided by the present invention are described in detail below with reference to the examples, but these examples should not be understood as limiting the claimed scope of the present invention.

### EXAMPLE 1

A Euglena culture medium, including the following components: 1.8 g/L NH₄Cl, 0.6 g/L KH₂PO₄, 1.2 g/L MgSO₄.7H₂O, 0.02 g/L CaCl₂).2H₂O, 0.55 μg/L Na₂EDTA.2H₂O, 2 μg/L Fe₂(SO₄)₃, 0.05 μg/L CuSO₄.5H₂O, 0.4 μg/L ZnSO₄.7H₂O, 1.3 μg/L Co (NH₃).H₂O, 1.8 μg/L MnCl₂.4H₂O, 0.01 μg/L of vitamin B₁ and 0.0005 μg/L of vitamin B₁₂.

Table 1 shows the main ions composition of the Euglena culture medium prepared by Example 1, where the main metal ions are Mg²⁺ and Ca²⁺.

TABLE 1
Concentrations of the main ions (>1 mg/L)
in Euglena culture medium

| Ions | mg/L | mM |
|---|---|---|
| Cl⁻ | 1199.64 | 33.84 |
| NH₄⁺ | 605.72 | 33.65 |
| SO₄²⁻ | 479.72 | 5.00 |
| PO₄³⁻ | 418.84 | 4.41 |
| K⁺ | 171.95 | 4.41 |
| Mg²⁺ | 119.77 | 4.99 |
| Ca²⁺ | 7.21 | 0.18 |

### EXAMPLE 2

A Euglena culture medium, including the following components: 1.5 g/L NH₄Cl, 0.6 g/L KH₂PO₄, 1.2 g/L MgSO₄.7H₂O, 0.02 g/L CaCl₂).2H₂O, 0.55 μg/L Na₂EDTA.2H₂O, 2 μg/L Fe₂(SO₄)₃, 0.05 μg/L CuSO₄.5H₂O, 0.4 μg/L ZnSO₄.7H₂O, 1.2 μg/L Co.(NH₃).H₂O, 1.8 μg/L MnCl₂.4H₂O, 0.01 μg/L vitamin B₁ and 0.0005 μg/L vitamin B₁₂.

### EXAMPLE 3

A Euglena culture medium, including the following components: 2.7 g/L NH₄Cl, 2.4 g/L KH₂PO₄, 2.4 g/L MgSO₄.7H₂O, 0.10 g/L CaCl₂).2H₂O, 0.78 μg/L Na₂EDTA.2H₂O, 4 μg/L Fe₂(SO₄)₃, 0.08 μg/L CuSO₄.5H₂O, 0.7 μg/L ZnSO₄.7H₂O, 1.5 μg/L Co.(NH₃).H₂O, 2.2 μg/L MnCl₂.4H₂O, 0.09 μg/L vitamin B₁ and 0.0015 μg/L vitamin B₁₂.

### APPLICATION EXAMPLE 1

(1) Euglena was inoculated into the Euglena culture medium provided by Example 1, and cultured under a 25° C. sterile condition to obtain a Euglena solution with an optical density at an absorption wavelength of 750 nm, namely OD₇₅₀=1.

(2) The Euglena solution was transferred to a photobioreactor, and cultured for 7 d under condition with a light intensity of 200 μmol photons m⁻²·s⁻¹ and an aeration rate of 5 L/min to obtain a high-concentration Euglena solution.

(3) 45 mL of Euglena solution (OD₇₅₀=1) obtained in step (2) was added into a 50 mL centrifuge tube, and the pH of the Euglena culture medium was adjusted to 8, 9 and 10 with 3 M of NaOH and 1 M of HCl.

After the centrifuge tube was left to stand for 60 min, the flocculation efficiency (FE) of the center of the solution was recorded.

Most Euglena cells were flocculated and precipitated to the bottom of the centrifuge tube to form pre-concentrated Euglena.

The FE was calculated as follows:

$$FE = \frac{ODb - ODa}{ODb} \times 100 \%$$

where, ODb is an OD before flocculation, and ODa is an OD after flocculation.

### COMPARATIVE EXAMPLE 1

After the centrifuge tube was left to stand for 10, 20, 30, 40, and 50 min, the FE of the center of the solution was recorded. The rest of the treatment were the same as in Application Example 1.

### COMPARATIVE EXAMPLE 2

The pH of the Euglena culture medium with OD₇₅₀=1 was adjusted to 3, 4, 5, 6, 7, 11 and 12 with 3 M of NaOH and 1 M of HCl. After the centrifuge tube was left to stand for 10, 20, 30, 40, 50 and 60 min, the FE of the center of the solution was recorded. The rest were the same as in Application Example 1.

Table 2 shows the FE of Euglena with different pH after standing for different times. It can be seen from Table 2 that at the same flocculation time, the FE at pH 8-10 was higher than the FE at pH 3, 4, 5, 6, 7, 11 and 12. In Application Example 1, the FE was higher at pH 8-10 at the flocculation time of 60 min.

TABLE 2
FE of pre-concentrated Euglena at pH 3-12 at different times

| Time | pH | | | | | | | | | |
|---|---|---|---|---|---|---|---|---|---|---|
| (min) | 3 | 4 | 5 | 6 | 7 | 8 | 9 | 10 | 11 | 12 |
| 10 | 17.09 | 19.68 | 24.95 | 36.35 | 39.14 | 49.14 | 42.63 | 38.82 | 36.95 | 33.34 |
| 20 | 20.70 | 35.76 | 37.55 | 47.40 | 50.62 | 57.38 | 53.51 | 46.85 | 46.64 | 44.60 |
| 30 | 30.98 | 35.62 | 47.24 | 57.45 | 58.02 | 78.02 | 70.09 | 67.52 | 57.86 | 52.00 |
| 40 | 50.99 | 54.80 | 57.12 | 68.85 | 69.22 | 93.65 | 84.57 | 71.58 | 64.56 | 60.51 |
| 50 | 54.83 | 60.23 | 64.15 | 69.79 | 73.89 | 94.77 | 86.77 | 82.16 | 77.00 | 61.96 |
| 60 | 61.76 | 63.74 | 67.58 | 70.53 | 74.58 | 97.46 | 89.95 | 80.38 | 73.77 | 67.30 |

### APPLICATION EXAMPLE 2

The test was carried out using the method of Application Example 1, except that the pH of the OD₇₅₀=1 Euglena culture medium was adjusted to 8, 8.5 and 9 with 3 M of NaOH and 1 M of HCl.

### COMPARATIVE EXAMPLE 3

The test was carried out using the method of Application Example 2, except that the FE in the center of the solution in the centrifuge tube was recorded at 20 and 40 min, respectively.

The test results are shown in Table 3. It can be seen from Table 3 that at the same flocculation time, the FE was higher at pH 8.5 of the Euglena culture medium.

TABLE 3
FE of pre-concentrated Euglena at
pH 8, 8.5 and 9 at different times

| Time | pH | | |
|---|---|---|---|
| (min) | 8 | 8.5 | 9 |
| 20 | 63 | 73* | 66 |
| 40 | 78.5 | 88* | 84 |
| 60 | 92 | 98.75* | 91.25 |

Note:
*represents a 0.05 level of significance.

### APPLICATION EXAMPLE 3

Control Group: The test was carried out using the method of Application Example 1 except that the pH was 3.

Test Group: The pH of the Euglena culture medium with OD₇₅₀=1, 1.5, 2 and 2.5 was adjusted to 8.5 with 3 M of NaOH and 1 M of HCl. The rest were the same as in Application Example 1.

The test results of different ODs are shown in Table 4. The FE basically reached more than 89% under different ODs, and there was no significant difference.

TABLE 4
FE under different ODs

| | Time (min) | | |
|---|---|---|---|
| OD | 20 | 40 | 60 |
| Control Group | 39.25 | 43.25 | 46.15 |
| 1 | 64.62 | 79.12 | 92.12 |
| 1.5 | 65.62 | 81.21 | 89.62 |
| 2 | 72.67 | 83.71 | 89.62 |
| 2.5 | 75.17 | 86.71 | 92.62 |

### APPLICATION EXAMPLE 4

Treatment 1: The test was carried out using the method of Application Example 1 except that the pH was 3.5.

Treatment 2: The test was carried out using the method of Application Example 1 except that the pH was 8.5.

Treatment 3: The test was carried out using the method of Application Example 1 except that the pH was 10.5.

The Euglena pre-concentrated by the three treatments was left to stand for 60 min, and the cellular morphology was observed.

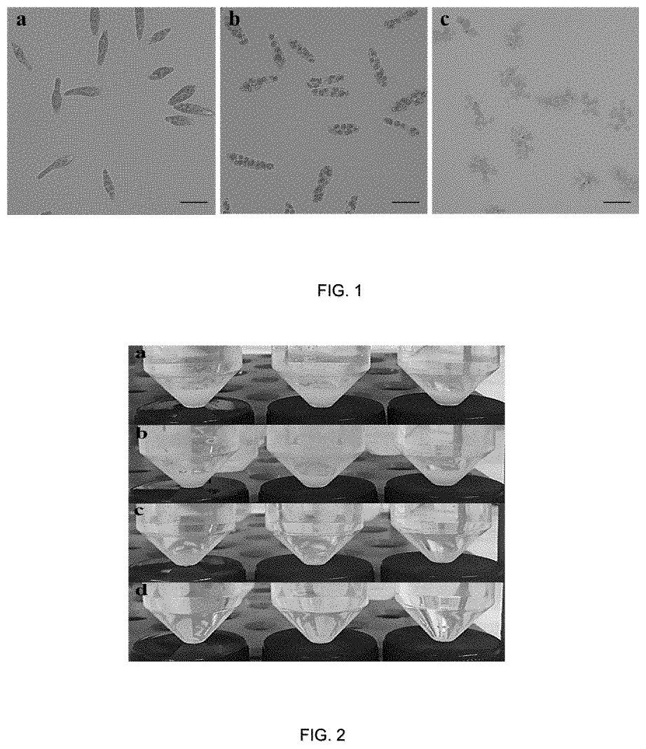

The test results are shown in FIG. 1, where a shows the cellular morphology of Euglena at pH 3.5 (Control group); b shows the cellular morphology of Euglena at pH 8.5; c shows the cellular morphology of Euglena at pH 10.5, with a scale bar being 20 μm. The changing cellular morphology shows that the cell had completed rupture at pH 10.5, but did not rupture at pH 3.5 and 8.5.

### TEST EXAMPLE 1

Treatment 1: The test was carried out using the method of Application Example 1, except that the Ca²⁺ of the culture medium in Example 1 was removed to obtain a comparative culture medium 1, and the pH was adjusted to 8.5.

Treatment 2: The test was carried out using the method of Application Example 1, except that the Mg²⁺ of the culture medium in Example 1 was removed to obtain a comparative culture medium 2, and the pH was adjusted to 8.5.

Treatment 3: The test was carried out using the method of Application Example 1, except that the Ca²⁺ and Mg²⁺ of the culture medium in Example 1 were removed to obtain a comparative culture medium 3, and the pH was adjusted to 8.5.

Treatment 4: The test was carried out using the method of Application Example 1 except that the pH was adjusted to 8.5.

Each treatment was repeated three times. The Euglena pre-concentrated by the four treatments was left to stand for 60 min, and the precipitation was observed.

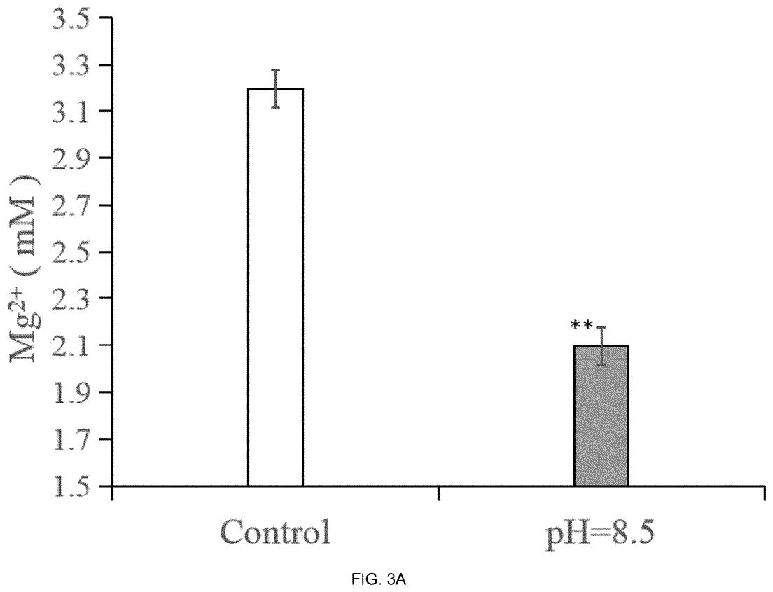

The test results are shown in FIG. 2, where a represents Treatment 4, b represents Treatment 1, c represents Treatment 2, and d represents Treatment 3. It was found that there was still a precipitate produced in the absence of Ca²⁺, while only a small amount of calcium hydroxide precipitate formed in the absence of Mg²⁺. When neither of these ions were present, no precipitate was formed, proving that the formation of the precipitate was caused by the action of these two metal ions.

### TEST EXAMPLE 2

In order to verify that these precipitates were responsible for the occurrence of flocculation, the following treatments were carried out:

Treatment 1: The test was carried out using the method of Application Example 1, except that the pH was 3.5, and the culture temperature in step (1) was 27° C.

Treatment 2: The test was carried out using the method of Application Example 1, except that the pH was 8.5, and the culture temperature in step (1) was 27° C.

Treatment 3: The test was carried out using the method of Application Example 1, except that a saturated chelating agent (10 M of ethylene diamine tetraacetic acid (EDTA)) was added to the Euglena culture medium in Example 1, the pH was 3.5, and the culture temperature in step (1) was 27° C.

Treatment 4: The test was carried out using the method of Application Example 1, except that a saturated chelating agent (10 M of EDTA) was added to the Euglena culture medium in Example 1, the pH was 8.5, and the culture temperature in step (1) was 27° C. The Euglena pre-concentrated by the four treatments was left to stand for 60 min, and the FE and Mg²⁺ and Ca²⁺ concentrations were measured.

The test results are shown in Table 5. According to Table 5, after the chelating agent was added, the PE was 41.63%, which was no different from the control group which did not adjust the pH, and when no EDTA was added, the PE reached 95.16% at pH 8.5.

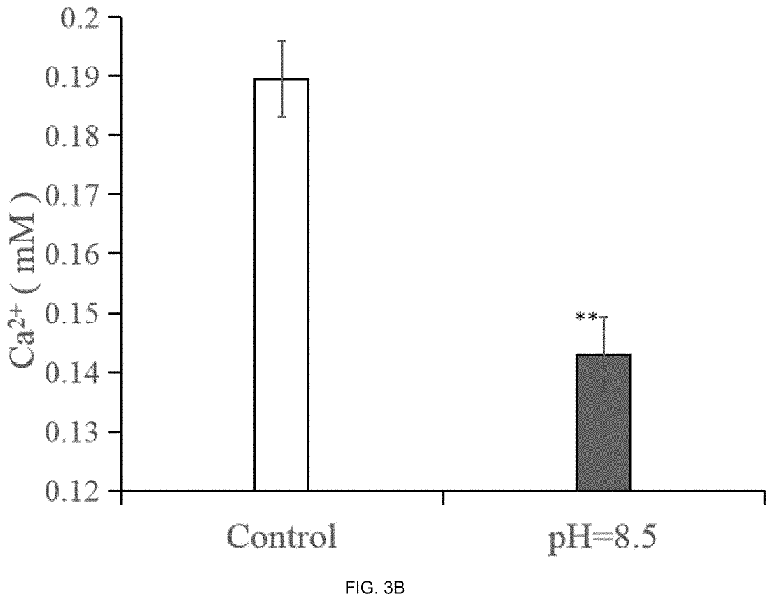

The concentrations of Mg²⁺ and Ca²⁺ are respectively shown in FIG. 3A and FIG. 3B, where the left graph shows the concentration of Mg²⁺ and the right graph shows the concentration of Ca²⁺. In the figure, “control” represents Treatment 1 and “pH=8.5” represents Treatment 2. The decrease in the concentrations of Mg²⁺ and Ca²⁺ was similar. As can be seen from the FE and concentration that the precipitate was the main cause for the flocculation of Euglena cells, and that the Euglena culture medium in Example 1 could improve the FE.

TABLE 5
Effect of chelating agent on flocculation

| Treatments | Treatment 1 | Treatment 2 | Treatment 3 | Treatment 4 |
|---|---|---|---|---|
| FE (%) | 42.87 | 95.16** | 41.63 | 41.99 |

Note:
**represents a 0.01 level of significance.

### TEST EXAMPLE 3

Treatment a1: The test was carried out using the Euglena culture medium in Example 1 and the method of Application Example 1, except that the pH was 3.5.

Treatment a2: The test was carried out using the Euglena culture medium in Example 1 and the method of Application Example 1, except that the pH was 8.5.

Treatment b1: The test was carried out using the Euglena culture medium in Example 1 and the method of Application Example 1, except that a magnesium ion with 2 times of concentration was added to the Euglena culture medium in Example 1 to obtain a comparative culture medium 4, and the pH was 3.5.

Treatment b2: The test was carried out using the Euglena culture medium in Example 1 and the method of Application Example 1, except that a magnesium ion with 2 times of concentration was added to the Euglena culture medium in Example 1 to obtain a comparative culture medium 5, and the pH was 8.5.

Treatment c1: The test was carried out using the Euglena culture medium in Example 1 and the method of Application Example 1, except that a magnesium ion with 4 times of concentration was added to the Euglena culture medium in Example 1 to obtain a comparative culture medium 6, and the pH was 3.5.

Treatment c2: The test was carried out using the Euglena culture medium in Example 1 and the method of Application Example 1, except that a magnesium ion with 4 times of concentration was added to the Euglena culture medium in Example 1 to obtain a comparative culture medium 7, and the pH was 8.5.

Treatment d1: The test was carried out using the Euglena culture medium in Example 1 and the method of Application Example 1, except that a calcium ion with 2 times of concentration was added to the Euglena culture medium in Example 1 to obtain a comparative culture medium 8, and the pH was 3.5.

Treatment d2: The test was carried out using the Euglena culture medium in Example 1 and the method of Application Example 1, except that a calcium ion with 2 times of concentration was added to the Euglena culture medium in Example 1 to obtain a comparative culture medium 9, and the pH was 8.5.

Treatment e1: The test was carried out using the Euglena culture medium in Example 1 and the method of Application Example 1, except that a calcium ion with 4 times of concentration was added to the Euglena culture medium in Example 1 to obtain a comparative culture medium 10, and the pH was 3.5.

Treatment e2: The test was carried out using the Euglena culture medium in Example 1 and the method of Application Example 1, except that a calcium ion with 4 times of concentration was added to the Euglena culture medium in Example 1 to obtain a comparative culture medium 11, and the pH was 8.5.

The Euglena pre-concentrated by the above treatments was left to stand for 60 min, and the FE and Mg²⁺ and Ca²⁺ concentrations were measured.

The test results are shown in Table 6 and Table 7. Table 6 shows that the soluble Mg²⁺ increased with the increasing added concentration, and Table 7 shows that the soluble Ca²⁺ does not change with the increasing added concentration, indicating that a large amount of Mg²⁺ could only form a small precipitate, while Ca²⁺ could form an effective precipitate. The flocculation was caused due to the combined action of Mg²⁺ and Ca²⁺, but Ca²⁺ played a key role.

TABLE 6
Concentration of Mg²⁺ in different treatments

| | Mg²⁺ Concentration | | |
|---|---|---|---|
| Treatments | Treatment (a1/a2) | Treatment (b1/b2) | Treatment (c1/c2) |
| pH = 3.5 | 5.01 | 8.22 | 18.98 |
| pH = 8.5 | 4.20 | 6.88 | 14.57 |

TABLE 7
Concentration of Ca²⁺ in different treatments

| | Ca²⁺ Concentration | | |
|---|---|---|---|
| Treatments | Treatment (a1/a2) | Treatment (b1/b2) | Treatment (c1/c2) |
| pH = 3.5 | 0.17 | 0.41 | 0.78 |
| pH = 8.5 | 0.12** | 0.14** | 0.17** |

Note:
**represents a 0.01 level of significance.

### COMPARATIVE EXAMPLE 2

Treatment A: The test was carried out using the Euglena culture medium in Example 1 and the method of Application Example 1, except that the pH was 8.5, and the culture temperature in step (1) was 29° C.

Treatment B: The test was carried out using the Euglena culture medium in Example 1 and the method of Application Example 1, except that the Ca²⁺ concentration in the Euglena culture medium of Example 1 was adjusted to 4 times, the pH was 8.5, and the culture temperature in step (1) was 29° C.

Treatment C: The test was carried out using the Euglena culture medium in Example 1 and the method of Application Example 1, except that the Mg²⁺ concentration in the Euglena culture medium of Example 1 was adjusted to 4 times, the pH was 8.5, and the culture temperature in step (1) was 29° C.

Treatment D: The test was carried out using the Euglena culture medium in Example 1 and the method of Application Example 1, except that the Ca²⁺ concentration in the Euglena culture medium of Example 1 was adjusted to 4 times, potassium hydrogen phosphate was added, the pH was 8.5, and the culture temperature in step (1) was 29° C.

Treatment E: The test was carried out using the Euglena culture medium in Example 1 and the method of Application Example 1, except that the Mg²⁺ concentration in the Euglena culture medium of Example 1 was adjusted to 4 times, potassium hydrogen phosphate was added, the pH was 8.5, and the culture temperature in step (1) was 29° C.

The Euglena pre-concentrated by the five treatments was left to stand for 60 min, and the FE was measured.

The test results are shown in Table 8. In the absence of PO4³⁻, the FE in the Euglena solution had no difference from that in the control group; in the presence of PO4³⁻, due to the effect of Ca²⁺ and Mg²⁺ with 4 times of concentration, the FE was 100% and 62.98%, respectively. This indicates that a large amount of calcium phosphate was produced at pH=8.5 of the Euglena culture medium obtained in Example 1, proving that the calcium phosphate precipitate was the main cause of flocculation and precipitation of Euglena cells.

TABLE 8
FE in different treatments

| | Treatments | | | | |
|---|---|---|---|---|---|
| | Treatment | Treatment | Treatment | Treatment | Treatment |
| | A | B | C | D | E |
| FE (%) | 35.45 | 42.24 | 43.12 | 100 | 62.98** |

Note:
* represents a 0.05 level of significance, and
**represents a 0.01 level of significance.

### APPLICATION EXAMPLE 5

The test was carried out using the Euglena culture medium in Example 1 and the method of Application Example 1, except that step (3) was carried out in a photobioreactor, and the pH was 8.5.

After the centrifuge tube was left to stand for 60 min, 90 min and 120 min, the FE of the center of the solution was recorded.

### COMPARATIVE EXAMPLE 3

The test was carried out using the Euglena culture medium in Example 1 and the method of Application Example 1, except that step (3) was carried out in a photobioreactor, and the pH was 8.5. After the centrifuge tube was left to stand for 30 min, the FE of the center of the solution was recorded.

### COMPARATIVE EXAMPLE 4

The test was carried out using the Euglena culture medium in Example 1 and the method of Application Example 1, except that step (3) was carried out in a photobioreactor, and the pH was 3.5. After the centrifuge tube was left to stand for 30 min, 60 min, 90 min and 120 min, the FE of the center of the solution was recorded.

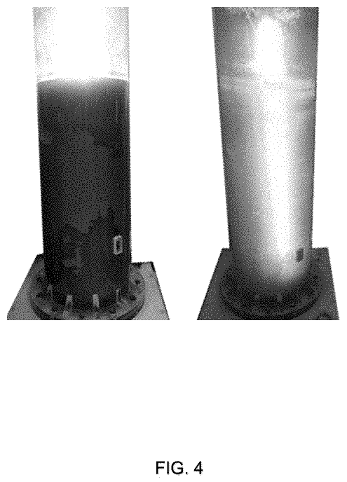

The flocculation effect of the Euglena culture medium with pH=8.5 after standing for 120 min is shown in FIG. 4. In the figure, the left picture shows the flocculation before the pH was adjusted, and the right picture shows the flocculation of the Euglena culture medium with pH=8.5 after standing for 120 min. The comparison shows that the Euglena culture medium provided in Example 1 could cause the Euglena cells to flocculate at pH 8.5 to obtain pre-concentrated Euglena.

Table 9 shows the FE of the Euglena culture medium with different pH after treatment and standing for different times. As can be seen from Table 9 that the FE of the Euglena culture medium at pH=8.5 was higher than that at pH=3.5, and the FE after standing for 60 min, 90 min and 120 min in Application Example 5 was higher than that after standing for 30 min in Comparative Example 3.

TABLE 9
FE after standing for different times

Standing Time (min)

| Treatments | 30 | 60 | 90 | 120 |
|---|---|---|---|---|
| pH = 3.5 | 12.98 | 23.9 | 34.1 | 32.9 |
| pH = 8.5 | 30 | 53.12 | 78.2 | 82.4 |

The above examples indicate that by using the Euglena culture medium of the present invention to concentrate a Euglena solution, Euglena is less ruptured, which improves the concentration efficiency of Euglena cells and reduces the treatment cost.

Although the aforementioned examples illustrate the present invention in detail, they are only parts of the examples of the present invention, rather than all of the examples. Other examples can be obtained by people according to these examples without the premise of inventiveness, and all of the examples fall within the claimed scope of the present invention.

What is claimed is:

1. A Euglena culture medium, comprising the following components: NH₄Cl, KH₂PO₄, MgSO₄.7H₂O, CaCl₂).2H₂O, Na₂EDTA.2H₂O, Fe₂(SO₄)₃, CuSO₄.5H₂O, ZnSO₄.7H₂O, Co.(NH₃).H₂O, MnCl₂.4H₂O, vitamin B₁ and vitamin B₁₂.

2. The Euglena culture medium according to claim 1, comprising the following components:1.5-2.7 g/L NH₄Cl, 0.6-2.4 g/L KH₂PO₄, 1.2-2.4 g/L MgSO₄.7H₂O, 0.02-0.10 g/L CaCl₂).2H₂O, 0.55-0.78 μg/L Na₂EDTA.2H₂O, 2-4 μg/L Fe₂(SO₄)₃, 0.05-0.08 μg/L CuSO₄.5H₂O, 0.4-0.7 μg/L ZnSO₄.7H₂O, 1.2-1.5 μg/L Co.(NH₃).H₂O, 1.8-2.2 μg/L MnCl₂.4H₂O, 0.01-0.09 μg/L vitamin B₁ and 0.0005-0.0015 μg/L vitamin B₁₂.

3. An application of the Euglena culture medium according to claim 1 in Euglena pre-concentration.

4. An application of the Euglena culture medium according to claim 2 in Euglena pre-concentration.

5. The application according to claim 3, wherein the Euglena comprises Euglena gracilis.

6. The application according to claim 4, wherein the Euglena comprises Euglena gracilis.

7. The application according to claim 3, comprising the following steps:
(1) inoculating Euglena into the Euglena culture medium to obtain a secondary Euglena solution;
(2) transferring the secondary Euglena solution to a photobioreactor, and continuing culture to obtain a Euglena solution; and
(3) adjusting pH of the Euglena solution to 8-10, and leaving the Euglena solution to stand to obtain pre-concentrated Euglena.

8. The application according to claim 4, comprising the following steps:
(1) inoculating Euglena into the Euglena culture medium to obtain a secondary Euglena solution;
(2) transferring the secondary Euglena solution to a photobioreactor, and continuing culture to obtain a Euglena solution; and
(3) adjusting pH of the Euglena solution to 8-10, and leaving the Euglena solution to stand to obtain pre-concentrated Euglena.

9. The application according to claim 7, wherein in step (3), a pH adjusting agent comprises 2-4 M of sodium hydroxide and 0.5-1.5 M of hydrochloric acid, and the standing time is preferably 60-120 min.

10. The application according to claim 8, wherein in step (3), a pH adjusting agent comprises 2-4 M of sodium hydroxide and 0.5-1.5 M of hydrochloric acid, and the standing time is preferably 60-120 min.

11. The application according to claim 7, wherein in step (2), the culture is performed under a condition with a light intensity of 100-200 μmol photons m⁻²·s⁻¹ and an aeration rate of 3-7 L/min.

12. The application according to claim 8, wherein in step (2), the culture is performed under a condition with a light intensity of 100-200 μmol photons m⁻²·s⁻¹ and an aeration rate of 3-7 L/min.

13. The application according to claim 11, wherein a gas used to maintain the aeration rate comprises carbon dioxide;
the volume content of the carbon dioxide in the gas is 1-3%; the carbon dioxide is carbon dioxide filtered through a 0.2 μm membrane.

14. The application according to claim 12, wherein a gas used to maintain the aeration rate comprises carbon dioxide;
the volume content of the carbon dioxide in the gas is 1-3%; the carbon dioxide is carbon dioxide filtered through a 0.2 μm membrane.

15. The application according to claim 7, wherein in step (2), the culture time is 6-8 d.

16. The application according to claim 8, wherein in step (2), the culture time is 6-8 d.

17. The application according to claim 11, wherein in step (2), the culture time is 6-8 d.

18. The application according to claim 12, wherein in step (2), the culture time is 6-8 d.

19. The application according to claim 13, wherein in step (2), the culture time is 6-8 d.

20. The application according to claim 7, wherein in step (1), the culture is performed under a sterile condition at 20-30° C. and a light intensity of 30-70 μmol photons m⁻²·s⁻¹.
